(19) United States
(12) Patent Application Publication
(10) Pub. No.: US 2024/0112337 A1
(43) Pub. Date: Apr. 4, 2024

(54) VEHICULAR DRIVER MONITORING SYSTEM WITH HEALTH MONITORING

(71) Applicant: Magna Electronics Inc., Auburn Hills, MI (US)

(72) Inventor: Sylvie Wacquant, Mainhausen (DE)

(21) Appl. No.: 18/480,531

(22) Filed: Oct. 4, 2023

Related U.S. Application Data
(60) Provisional application No. 63/378,313, filed on Oct. 4, 2022.

(51) Int. Cl. G06T 7/00; A61B 5/00; B60Q 9/00; B60W 30/09; B60W 30/12; B60W 30/14; B60W 50/08; G06V 20/52; G06V 20/59; G08G 1/16

(52) U.S. Cl. CPC G06T 7/0012; A61B 5/0077; B60Q 9/00; B60W 30/09; B60W 30/12; B60W 30/14; B60W 50/085; G06V 20/52; G06V 20/597; G08G 1/167; A61B 2503/22; B60W 2420/42; B60W 2540/221; B60W 2540/229; B60W 2540/24; B60W 2555/60; B60W 2556/45; B60W 2710/18; G06T 2207/20081; G06T 2207/30196; G06T 2207/30232; G06T 2207/30268

(57) ABSTRACT

A vehicular driver monitoring system includes a camera that views the driver of a vehicle. The vehicular driver monitoring system accesses a driver profile associated with the driver that includes status of a health parameter of the driver. The vehicular driver monitoring system, responsive at least in part to processing at an electronic control unit (ECU) of image data captured by the camera, determines a current status of the health parameter of the driver. The driver profile associated with the driver includes at least one previously determined status of the health parameter of the driver. The vehicular driver monitoring system determines a health rating of the driver based on a comparison of (i) the determined current status of the health parameter of the driver and (ii) the at least one previously determined status of the health parameter of the driver.

### CROSS REFERENCE TO RELATED APPLICATION

The present application claims the filing benefits of U.S. provisional application Ser. No. 63/378,313, filed Oct. 4, 2022, which is hereby incorporated herein by reference in its entirety.

### FIELD OF THE INVENTION

The present invention relates generally to a monitoring system for a vehicle and, more particularly, to a vehicular driver monitoring system or a vehicular occupant monitoring system that utilizes one or more cameras at a vehicle.

### BACKGROUND OF THE INVENTION

Use of imaging sensors in vehicle imaging systems is common and known. Examples of such known systems are described in U.S. Pat. Nos. 5,949,331; 5,670,935 and/or 5,550,677, which are hereby incorporated herein by reference in their entireties.

### SUMMARY OF THE INVENTION

A vehicular driver monitoring system includes a camera disposed within an interior cabin of a vehicle equipped with the vehicular driver monitoring system. The camera views a driver of the vehicle and is operable to capture image data. The camera includes a CMOS imaging array with at least one million photosensors arranged in rows and columns. The system includes an electronic control unit (ECU) with electronic circuitry and associated software. Image data captured by the camera is processed at the ECU. The vehicular driver monitoring system accesses a driver profile associated with the driver of the vehicle that includes status of a health parameter of the driver of the vehicle. The vehicular driver monitoring system, responsive at least in part to processing at the ECU of image data captured by the camera, determines a current status of the health parameter of the driver of the vehicle. The driver profile associated with the driver of the vehicle includes at least one previously determined status of the health parameter of the driver of the vehicle. The vehicular driver monitoring system updates the driver profile associated with the driver of the vehicle based on the determined current status of the health parameter of the driver of the vehicle. The vehicular driver monitoring system determines a health rating of the driver of the vehicle based on a comparison of (i) the determined current status of the health parameter of the driver of the vehicle and (ii) the at least one previously determined status of the health parameter of the driver of the vehicle.

These and other objects, advantages, purposes and features of the present invention will become apparent upon review of the following specification in conjunction with the drawings.

### BRIEF DESCRIPTION OF THE DRAWINGS

FIG. 1 is a plan view of a vehicle with a driver monitoring system that incorporates one or more sensors;

FIG. 2 is a timeline of the driver monitoring system of FIG. 1 capturing biometric data;

FIG. 3 is a schematic view of exemplary health monitoring devices for the driver monitoring system of FIG. 1;

FIG. 4 is an exemplary flow chart for the driver monitoring system of FIG. 1; and

FIG. 5 is a block diagram for an analyzer of the driver monitoring system of FIG. 1.

### DESCRIPTION OF THE PREFERRED EMBODIMENTS

A vehicular driver monitoring system and/or a vehicular occupant monitoring system operates to capture sensor data from one or more sensors sensing one or more occupants of a vehicle. The monitoring system includes a processor or processing system that is operable to receive sensor data from then sensors to determine one or more health values representative of a measure of one or more health parameters of the occupants. The system may include a display for displaying warnings or other notifications based on processing of the health values of the occupants.

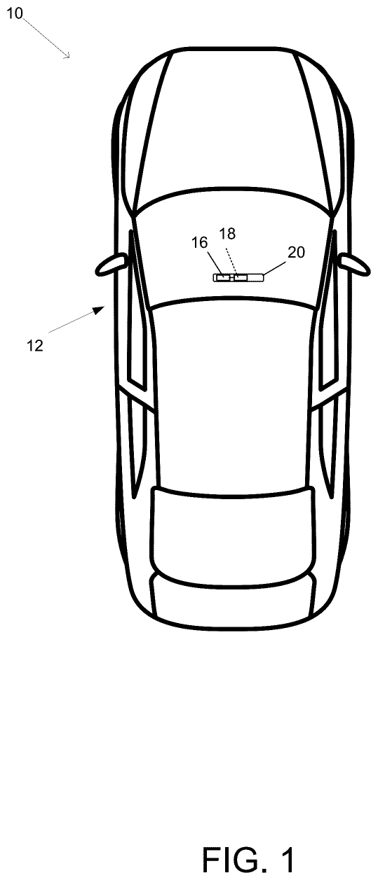

Referring now to the drawings and the illustrative embodiments depicted therein, a vehicle 10 includes a monitoring system 12 that includes at least one occupant sensor. For example, the occupant sensor include an interior camera 16, which captures images interior of the vehicle (e.g., of the driver or other occupants), with the camera having a lens for focusing images at or onto an imaging array or imaging plane or imager of the camera (FIG. 1). Although the camera 16 is shown in FIG. 1 as being part of or incorporated in or at an interior rearview mirror assembly 20 of the vehicle, the camera and/or other sensors may be disposed elsewhere at or in the vehicle. The monitoring system 12 includes a control or electronic control unit (ECU) 18 having electronic circuitry and associated software, with the electronic circuitry including a data processor or image processor that is operable to process image data captured by the camera or other sensors, whereby the ECU may determine one or more health values or statuses of occupants of the vehicle (e.g., heart rate values, breathing values, drowsiness values, etc.). The data transfer or signal communication from the sensors to the ECU may comprise any suitable data or communication link, such as a vehicle network bus or the like of the equipped vehicle.

Due to the average age of many countries increasing, overall population health (e.g., increases in chronic diseases), and lack of sufficient healthcare personnel, prevention has become a primary focus of successful healthcare. Successful prevention generally includes the regular monitoring of people. Conventional monitoring for healthcare professionals often involves providing a device to a patient for a period of time and hoping that the patient uses the device properly to enable health analysis. Regular health monitoring is often beneficial for any age group and/or demographic (e.g., children).

A significant number of people drive or ride in a vehicle daily for at least 15 minutes. For example, some studies have shown that average citizens in some areas of the world spend, on average, an hour per day in a vehicle. Implementations herein include a vehicular driver monitoring system and/or a vehicular occupant monitoring system that uses this time to determine any number of typical health values/parameters to determine or estimate or predict a health state of the driver and/or occupant of the vehicle. The system may react quickly in response to early signs of a disease (e.g., chronic diseases) and/or other health conditions. By performing this monitoring in the vehicle, the occupants may receive regular health monitoring without the need to sacrifice time elsewhere in their schedules. Thus, the system makes efficient use of the occupant's time while obtaining monitoring data at regular intervals within a consistent environment.

Optionally, the monitoring system determines whether the driver of the vehicle is healthy enough to safely operate the vehicle. The system may track any number of health parameters or values or statuses or health criteria for each occupant of the vehicle individually. The system may keep a health profile for each individual (e.g., stored at memory disposed within the vehicle or stored on a remote server in communication with the vehicle, such as via the Internet) and update the appropriate profile whenever the individual is an occupant of the vehicle. The system may store a plurality of profiles and may identify the occupant in the vehicle (such as via processing of image data captured by the drier monitoring camera) and use the profile stored for that identified occupant.

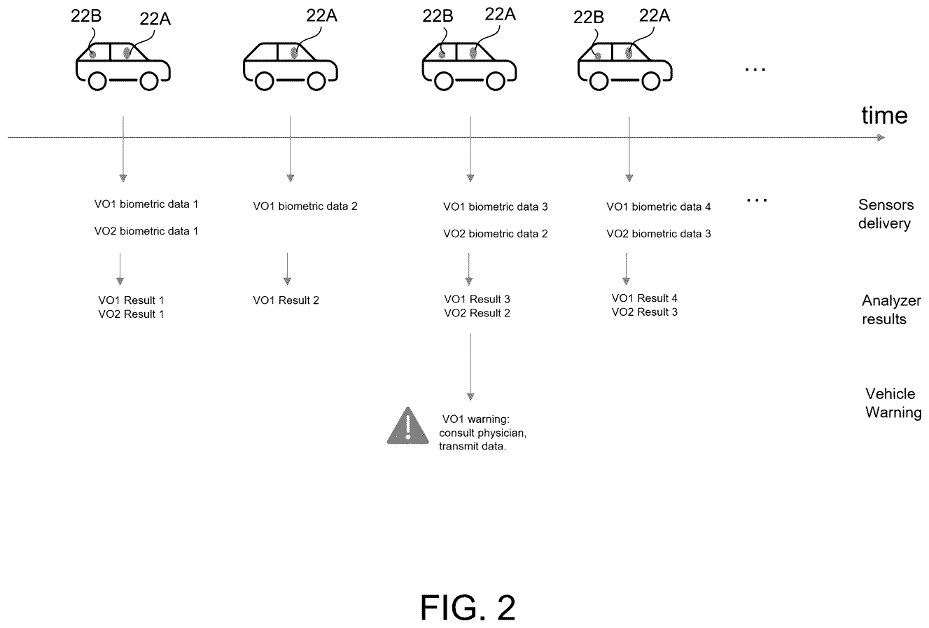

For example, and referring now to FIG. 2, at a first time interval, a vehicle includes a first occupant 22A (e.g., a driver of the vehicle) and a second occupant 22B. In this instance, the system captures and stores biometric data/health data for the first vehicle occupant (VO) 22A with a first profile/result associated with the first occupant 22A (i.e., “VO1 Result 1”). Similarly, the system captures and stores biometric data/health data for the second occupant 22B with a second profile/result associated with the second occupant 22B (i.e., “VO2 Result 1”). In a subsequent time interval, only the first occupant 22A is within the vehicle (i.e., the second occupant 22B is not present). In this instance, the system captures and stores second biometric data for the first occupant (“VO1 Result 2”) and updates the first profile accordingly. In a third time interval, both the first occupant 22A and the second occupant 22B are present, and the system captures/stores third biometric data for the first occupant 22A (“VO1 Result 3”) and second biometric data for the second occupant 22B (“VO2 Result 2”). In this example, the system determines an anomaly in the biometric data sufficient to cause the system to generate a warning. For example, a health value satisfied a threshold value (e.g., exceeded/failed to exceed the threshold value/status or satisfied/failed to satisfy one or more health criteria or other complex evaluation), triggering the warning. Similarly, at a fourth time interval, both the first occupant 22A and the second occupant 22B are present within the vehicle and the system captures and stores fourth biometric data for the first occupant 22A (“VO1 Result 4”) and third biometric data for the second occupant 22B (“VO2 Result 3”). The system may continue to capture and store biometric data and update profiles/results accordingly for any number of individuals for any length of time. The system may identify the occupant(s) present in the vehicle via the driver monitoring system or occupant monitoring system, such as by processing image data captured by a cabin-viewing camera when the occupant enters the vehicle, or such as detecting the user's smartphone or other device (e.g., the driver's keyfob for the vehicle).

The system, when storing the biometric data and updating the profile/results, may compare the measured data with previously measured data and/or various data thresholds to determine a health rating of the particular individual based on a current status of a health parameter of the particular individual (e.g., an emotional state of the individual, a drowsiness level of the individual, a breath analysis of the individual, a skin analysis of the individual, a heart rate of the individual, etc.) and at least one previously determined status of the health parameter of the individual (e.g., multiple previously determined statuses determined via a previous trip or drive). For example, the system determines whether the measured data varies by a threshold amount (i.e., a threshold change) from previous measurements associated with the particular individual and/or varies by a threshold from nominal values (e.g., for an individual of the same age, gender, size, etc.). For example, the system determines that the determined health rating is indicative of a threshold change in a health parameter when the current status of the health parameters exceeds or is less than a threshold status of the health parameter.

The system may generate a notification that indicates a healthiness of the particular individual. For example, when the measured data varies by at least the threshold amount, the system may determine that the measured data is abnormal. When the system determines that the data is abnormal, the system may generate a warning or notification for the occupant that includes the health rating (e.g., indicating that the particular individual has an unhealthy health rating). The warning may be a visual warning displayed on a display within the vehicle and/or an audible warning. The warning may be sent to a user device of the occupant (e.g., via an email, text message, or an application executing on the user device). The warning may include instructions to seek additional care and/or the measured data. In some examples, the system may transmit the warning to a healthcare professional or to emergency services. For example, when the measured data is abnormal by a critical threshold amount, the system may automatically transmit applicable data to a healthcare professional (e.g., designated by the occupant) and/or emergency services. In other examples, the system automatically informs a healthcare professional after measuring abnormal data a threshold number of times.

Optionally, the system may determine when one or more health criteria are satisfied or have failed to be satisfied. A health criteria may refer to any simple or complex evaluation of the health value or status, such as patterns in the health value (e.g., within a first range of values for a first threshold period of time followed by being within a second range of values for a second threshold period of time) or the health value combined with other parameters (e.g., other health values, environmental conditions, or status of the occupant, such as age, gender, etc.). The system may evaluate the health values and/or health criteria locally (e.g., via an analyzer using local processing disposed at the vehicle). In other examples, the system may allow a remote server to evaluate the health values and/or health criteria. For example, the system may transmit the profile and/or health values to a remote server using, for example, wireless communication over a cellular data connection or via connection with a user device (e.g., a mobile phone). The remote server may evaluate the health values/health criteria and provide a response to the system indicating whether any thresholds are met and/or whether the health values indicate a healthiness or unhealthiness. Based on the response from the remote server, the system may update the associated profile and/or generate a notification for the occupant of the vehicle. The remote server may also transmit notifications directly to the user (e.g., to the user's mobile phone) or to healthcare professionals.

The system and/or the remote server may make use of any number of algorithms or machine learning models to analyze the health values and to make predictions or determinations. For example, a deep neural network may be trained to detect a healthiness or unhealthiness of health values for one or more occupants of the vehicle. The model may be trained on a fleet of data from many vehicles and provided to the remote server and/or vehicle to predict health conditions for the occupant. In some examples, the model may be further tuned or trained using the occupant's health values and/or other parameters associated with the occupant of the vehicle. The vehicle and/or remote server may host multiple models for different occupants/users of the vehicle. The system may receive feedback from the occupants/users and/or healthcare professionals on predictions/determinations to further refine or train the model. For example, when the model/algorithm predicts an unhealthy condition for an occupant, but the prediction is a false positive, this feedback may be provided to the model to refine/update the model. The model may learn from false positives and false negatives from both the occupant of the vehicle and/or from a fleet manager obtaining training data from other vehicles (e.g., predictions made by other models for other occupants at other vehicles).

Optionally, the system determines whether biometric data associated with an occupant improves (e.g., relative to nominal values or previous values of the occupant) after the occupant has sought treatment (e.g., medications). For example, the occupant and/or a healthcare professional may provide treatment information to the system, which the system uses to monitor treatment of the occupant. The system may monitor reactions of each occupant to external events (e.g., pollen allergy detection, alcohol levels, etc.). The system may determine whether the occupant remembers to take medication, performs some control or action, etc. The system may update/refine any models or algorithms based on results from the treatment.

When high precision measurements are not needed (i.e., only general monitoring is required), the system may rely on low cost devices directly available in the vehicle (e.g., cameras, radar sensors, or other sensors, such as other low cost sensors). In scenarios where an occupant requires high precision monitoring, a “medical vehicle” equipped with the system and necessary measuring devices may be rented to the occupant for regular monitoring during a certain time (weeks, months, years, etc.). Such medical vehicles may be available for “car sharing.”

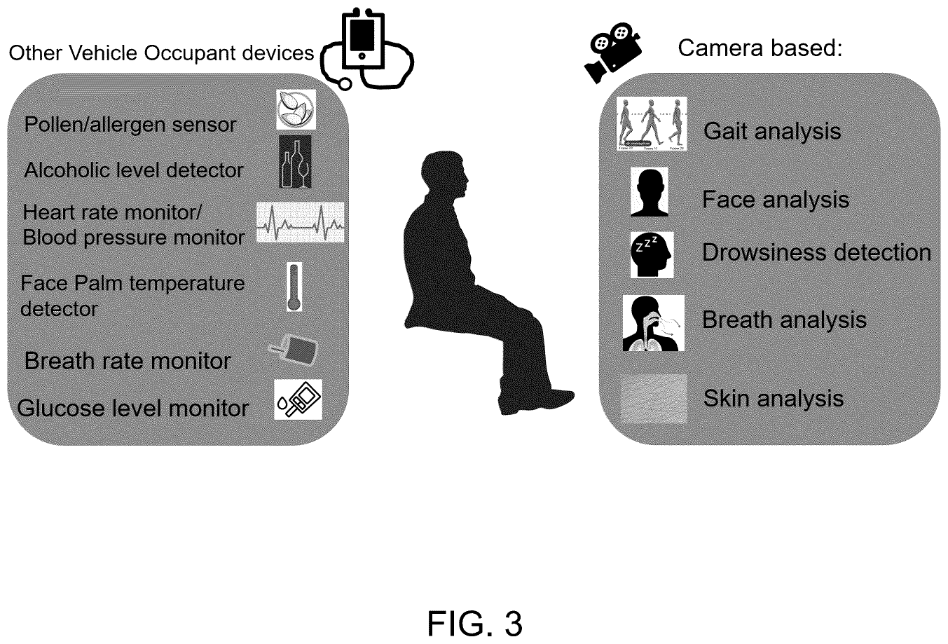

Referring now to FIG. 3, the system may receive health data or biometric data from any number of sensors disposed at or within the vehicle. For example, the system includes one or more cameras viewing the interior and/or exterior of the vehicle. For example, an occupant monitoring camera may be disposed at a rearview mirror of the vehicle and viewing the cabin of the vehicle. The occupant monitoring camera may comprise a cabin monitoring camera or a driver monitoring camera or the like. The camera may be disposed at a mirror head of the interior rearview mirror assembly, such as disposed behind the mirror reflective element and viewing through the mirror reflector of the mirror reflective element toward the interior cabin of the vehicle (with the camera moving together and in tandem with the mirror head when the driver adjusts his or her rearward view provided by the mirror reflective element). The camera (or cameras) thus may view the driver or occupant (or occupants) of the vehicle and may capture image data of the occupants, and the system may process and/or analyze the captured image data for a number of health purposes. For example, the system may perform gait analysis detection (e.g., by analyzing a gait of the occupant using image data captured by an exterior viewing camera of the occupant approaching or leaving the vehicle) to detect neurological diseases such as multiple sclerosis and/or Parkinson's disease and/or systemic diseases such as cardiopathies or alterations in deambulation dynamic due to sequelae from stroke and/or diseases caused by aging. The system may perform facial analysis (with or without breath/heart rate detection), detection of emotional dysfunctions (e.g., depression) based on emotions regularly detected, and/or drowsiness (e.g., via sagging posture, leaning of the occupant's head, status of the occupant's eyes, etc.). The system may perform breath analysis via image analysis (e.g., by vertical motion variations) or via a breath sensor disposed at the seat/seat belt of the vehicle. The system may perform heart rate analysis using, for example, a heart rate sensor disposed at the seat or seat belt or based on a wearable device of the occupant (e.g., a watch). The system may perform skin analysis using image data to determine potential presence of skin diseases, allergies, infections, cancer, etc.

The system may use a number of other sensors in addition to or alternative to one or more cameras. For example, the system may perform pollen/allergen monitoring (e.g., via one or more pollen sensors), alcoholic level detection (e.g., via a blood alcohol analyzer such as a fuel cell breathalyzer or infrared sensors), heart rate monitoring/blood pressure monitoring (e.g., via a heart rate monitoring seat or a device integrated into the steering wheel, seat belt, etc.), breath rate monitoring, glucose level monitoring, etc.

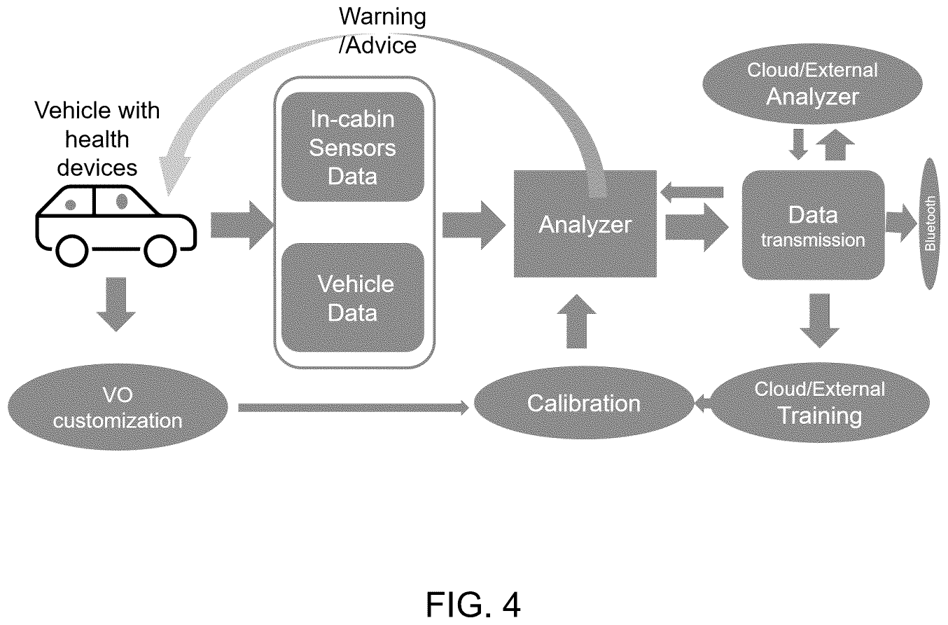

Referring now to FIG. 4, the measurements (i.e., the health data/biometric data) is captured by one or more sensors at or within the vehicle. This data is received by an analyzer (e.g., executed by a vehicle ECU or other processor) that processes and/or records the data. The data may be analyzed and compared to certain thresholds or profiles of individual occupants either configured by health experts (e.g., a healthcare professional of the occupant or medically accepted norms) and/or learned by the system (e.g., via machine learning), as well as customized by the profile of the occupant (e.g., based on previous measurements of the occupant and/or parameters of the occupant such as size, age, gender, etc.). The analyzer may compare measurements to previous measurements and/or thresholds. The system may compare the measurements to one or more health criteria associated with the measurements. The analyzer may include a machine learning medical diagnosis detector for processing the captured measurements. The detector may include any number of algorithms, such as a random forest algorithm, or a support vector machine (SVM), etc. In some examples (e.g., when the analysis is sufficiently complex), the system may upload the data to a remote server (e.g., the “cloud”) or other external device for further processing. The data may be returned back to the analyzer after processing for notification of the occupant. In some examples, the remote server may directly notify the occupant (e.g., via a user device such as a mobile phone).

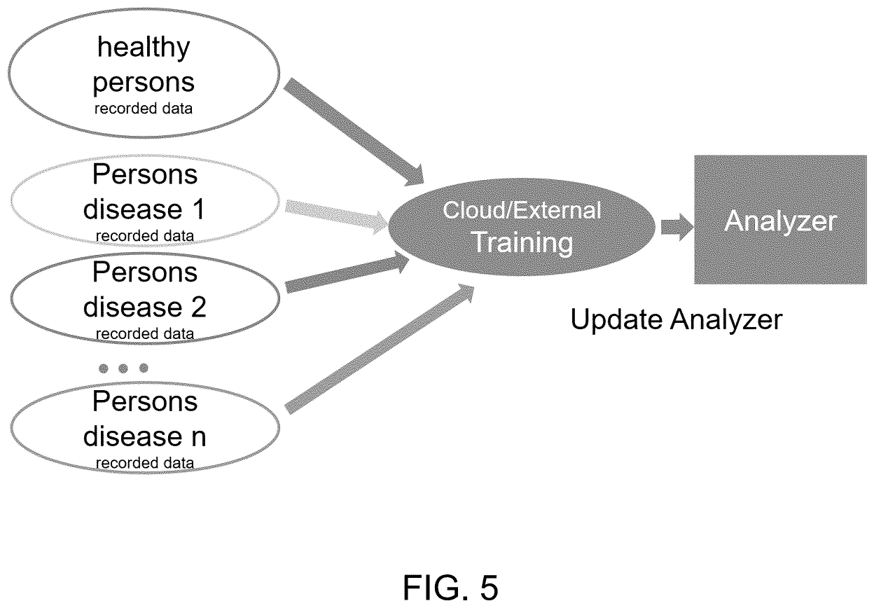

The system may anonymize the data prior to transmitting the data to a remote source. For example, anonymized data may be sent to healthcare professionals or experts (e.g., via the Internet using wireless communication such as cellular technologies, WIFI, or BLUETOOTH) to enable training or other improvements (FIG. 4). For example, the anonymized data may help determine thresholds and profiles or for training the detection algorithms (FIG. 5). The training may be separate for different algorithms/machines with different types of measurements/health data as needed.

Thus, in some implementations, the vehicular driver monitoring system stores in memory a driver profile associated with the driver, with the driver profile including a threshold value for a health parameter (e.g., a maximum heartrate or breathing rate, or a maximum stress level or a minimum attentiveness level, etc.) of the driver of the vehicle. The vehicular driver monitoring system, responsive at least in part to processing at the ECU of captured data, determines a current value for the health parameter of the driver of the vehicle. The driver profile associated with the driver includes at least one previously determined value for the health parameter of the driver of the vehicle, and the vehicular driver monitoring system updates the driver profile associated with the driver based on the determined current value for the health parameter of the driver of the vehicle. The vehicular driver monitoring system may determine a health rating of the driver based on a comparison of (i) the threshold value for the health parameter of the driver of the vehicle, (ii) the determined current value for the health parameter of the driver of the vehicle and (iii) the at least one previously determined value for the health parameter of the driver of the vehicle. The vehicular driver monitoring system may generate a health notification for the driver responsive to the determined health rating of the driver being indicative of a threshold change in the health parameter of the driver of the vehicle.

The ECU may be located at or within the interior rearview mirror assembly, such as in the mirror head or the mirror base. Optionally, the ECU may be located remote from the interior rearview mirror assembly. If the ECU is located remote from the interior rearview mirror assembly, the image data captured by the camera may be transferred to the ECU (and optionally control signals and/or electrical power from the ECU may be transferred to the camera) via a coaxial cable, such as by utilizing aspects of the systems described in U.S. Pat. Nos. 11,792,360; 11,638,070; 11,533,452; 11,508,160; 11,308,718; 11,290,679; 11,252,376; 11,201,994; 11,025,859; 10,922,563; 10,827,108; 10,694,150; 10,630,940; 10,567,705; 10,567,633; 10,515,279; 10,284,764; 10,089,537; 10,071,687; 10,057,544 and/or 9,900,490, which are all hereby incorporated herein by reference in their entireties.

The ECU may be operable to process data for at least one driving assist system of the vehicle. For example, the ECU may be operable to process data (such as image data captured by a forward viewing camera of the vehicle that views forward of the vehicle through the windshield of the vehicle) for at least one selected from the group consisting of (i) a headlamp control system of the vehicle, (ii) a pedestrian detection system of the vehicle, (iii) a traffic sign recognition system of the vehicle, (iv) a collision avoidance system of the vehicle, (v) an emergency braking system of the vehicle, (vi) a lane departure warning system of the vehicle, (vii) a lane keep assist system of the vehicle, (viii) a blind spot monitoring system of the vehicle and (ix) an adaptive cruise control system of the vehicle. Optionally, the ECU may also or otherwise process radar data captured by a radar sensor of the vehicle or other data captured by other sensors of the vehicle (such as other cameras or radar sensors or such as one or more lidar sensors of the vehicle). Optionally, the ECU may process captured data for an autonomous control system of the vehicle that controls steering and/or braking and/or accelerating of the vehicle as the vehicle travels along the road.

The system may utilize aspects of driver monitoring systems and/or head and face direction and position tracking systems and/or eye tracking systems and/or gesture recognition systems. Such head and face direction and/or position tracking systems and/or eye tracking systems and/or gesture recognition systems may utilize aspects of the systems described in U.S. Pat. Nos. 11,518,401; 10,958,830; 10,065,574; 10,017,114; 9,405,120 and/or 7,914,187, and/or U.S. Publication Nos. US-2022-0377219; US-2022-0254132; US-2022-0242438; US-2021-0323473; US-2021-0291739; US-2020-0320320; US-2020-0202151; US-2020-0143560; US-2019-0210615; US-2018-0231976; US-2018-0222414; US-2017-0274906; US-2017-0217367; US-2016-0209647; US-2016-0137126; US-2015-0352953; US-2015-0296135; US-2015-0294169; US-2015-0232030; US-2015-0092042; US-2015-0022664; US-2015-0015710; US-2015-0009010 and/or US-2014-0336876, and/or International Publication Nos. WO 2022/241423; WO 2022/187805 and/or WO 2023/034956, and/or PCT Application No. PCT/US2023/021799, filed May 11, 2023 (Attorney Docket DON01 FP4810WO), which are all hereby incorporated herein by reference in their entireties.

The camera or sensor may comprise any suitable camera or sensor. Optionally, the camera may comprise a “smart camera” that includes the imaging sensor array and associated circuitry and image processing circuitry and electrical connectors and the like as part of a camera module, such as by utilizing aspects of the vision systems described in U.S. Pat. Nos. 10,099,614 and/or 10,071,687, which are hereby incorporated herein by reference in their entireties.

The system includes an image processor operable to process image data captured by the camera or cameras, such as for detecting objects or other vehicles or pedestrians or the like in the field of view of one or more of the cameras. For example, the image processor may comprise an image processing chip selected from the EYEQ family of image processing chips available from Mobileye Vision Technologies Ltd. of Jerusalem, Israel, and may include object detection software (such as the types described in U.S. Pat. Nos. 7,855,755; 7,720,580 and/or 7,038,577, which are hereby incorporated herein by reference in their entireties), and may analyze image data to detect vehicles and/or other objects. Responsive to such image processing, and when an object or other vehicle is detected, the system may generate an alert to the driver of the vehicle and/or may generate an overlay at the displayed image to highlight or enhance display of the detected object or vehicle, in order to enhance the driver's awareness of the detected object or vehicle or hazardous condition during a driving maneuver of the equipped vehicle.

The vehicle may include any type of sensor or sensors, such as imaging sensors or radar sensors or lidar sensors or ultrasonic sensors or the like. The imaging sensor of the camera may capture image data for image processing and may comprise, for example, a two dimensional array of a plurality of photosensor elements arranged in at least 640 columns and 480 rows (at least a 640×480 imaging array, such as a megapixel imaging array or the like), with a respective lens focusing images onto respective portions of the array. The photosensor array may comprise a plurality of photosensor elements arranged in a photosensor array having rows and columns. The imaging array may comprise a CMOS imaging array having at least 300,000 photosensor elements or pixels, preferably at least 500,000 photosensor elements or pixels and more preferably at least one million photosensor elements or pixels or at least three million photosensor elements or pixels or at least five million photosensor elements or pixels arranged in rows and columns. The imaging array may capture color image data, such as via spectral filtering at the array, such as via an RGB (red, green and blue) filter or via a red/red complement filter or such as via an RCC (red, clear, clear) filter or the like. The logic and control circuit of the imaging sensor may function in any known manner, and the image processing and algorithmic processing may comprise any suitable means for processing the images and/or image data.

The ECU may receive image data captured by a plurality of cameras of the vehicle, such as by a plurality of surround view system (SVS) cameras and a plurality of camera monitoring system (CMS) cameras and optionally one or more driver monitoring system (DMS) cameras. The ECU may comprise a central or single ECU that processes image data captured by the cameras for a plurality of driving assist functions and may provide display of different video images to a video display screen in the vehicle (such as at an interior rearview mirror assembly or at a central console or the like) for viewing by a driver of the vehicle. The system may utilize aspects of the systems described in U.S. Pat. Nos. 10,442,360 and/or 10,046,706, and/or U.S. Publication Nos. US-2021-0245662; US-2021-0162926; US-2021-0155167 and/or US-2019-0118717, and/or International Publication No. WO 2022/150826, which are all hereby incorporated herein by reference in their entireties.

The ECU may process data captured by various sensors, such as radar sensors or imaging radar sensors or lidar sensors or the like, to detect presence of and/or range to objects and/or other vehicles and/or pedestrians. The system may utilize aspects of the systems described in U.S. Pat. Nos. 10,866,306; 9,954,955; 9,869,762; 9,753,121; 9,689,967; 9,599,702; 9,575,160; 9,146,898; 9,036,026; 8,027,029; 8,013,780; 7,408,627; 7,405,812; 7,379,163; 7,379,100; 7,375,803; 7,352,454; 7,340,077; 7,321,111; 7,310,431; 7,283,213; 7,212,663; 7,203,356; 7,176,438; 7,157,685; 7,053,357; 6,919,549; 6,906,793; 6,876,775; 6,710,770; 6,690,354; 6,678,039; 6,674,895 and/or 6,587,186, and/or U.S. Publication Nos. US-2019-0339382; US-2018-0231635; US-2018-0045812; US-2018-0015875; US-2017-0356994; US-2017-0315231; US-2017-0276788; US-2017-0254873; US-2017-0222311 and/or US-2010-0245066, which are hereby incorporated herein by reference in their entireties.

The system may also communicate with other systems, such as via a vehicle-to-vehicle communication system or a vehicle-to-infrastructure communication system or the like. Such car2car or vehicle to vehicle (V2V) and vehicle-to-infrastructure (car2X or V2X or V2I or a 4G or 5G broadband cellular network) technology provides for communication between vehicles and/or infrastructure based on information provided by one or more vehicles and/or information provided by a remote server or the like. Such vehicle communication systems may utilize aspects of the systems described in U.S. Pat. Nos. 10,819,943; 9,555,736; 6,690,268; 6,693,517 and/or 7,580,795, and/or U.S. Publication Nos. US-2014-0375476; US-2014-0218529; US-2013-0222592; US-2012-0218412; US-2012-0062743; US-2015-0251599; US-2015-0158499; US-2015-0124096; US-2015-0352953; US-2016-0036917 and/or US-2016-0210853, which are hereby incorporated herein by reference in their entireties.

Changes and modifications in the specifically described embodiments can be carried out without departing from the principles of the invention, which is intended to be limited only by the scope of the appended claims, as interpreted according to the principles of patent law including the doctrine of equivalents.

1. A vehicular driver monitoring system, the vehicular driver monitoring system comprising:
a camera disposed within an interior cabin of a vehicle equipped with the vehicular driver monitoring system, the camera viewing a driver of the vehicle, wherein the camera is operable to capture image data;
wherein the camera comprises a CMOS imaging array, and wherein the CMOS imaging array comprises at least one million photosensors arranged in rows and columns;
an electronic control unit (ECU) comprising electronic circuitry and associated software;
wherein image data captured by the camera is processed at the ECU;
wherein the vehicular driver monitoring system accesses a driver profile associated with the driver of the vehicle, and wherein the driver profile includes status of a health parameter of the driver of the vehicle;
wherein the vehicular driver monitoring system, responsive at least in part to processing at the ECU of image data captured by the camera, determines a current status of the health parameter of the driver of the vehicle;
wherein the driver profile associated with the driver of the vehicle comprises at least one previously determined status of the health parameter of the driver of the vehicle;
wherein the vehicular driver monitoring system updates the driver profile associated with the driver of the vehicle based on the determined current status of the health parameter of the driver of the vehicle; and
wherein the vehicular driver monitoring system determines a health rating of the driver of the vehicle based on a comparison of (i) the determined current status of the health parameter of the driver of the vehicle and (ii) the at least one previously determined status of the health parameter of the driver of the vehicle.

2. The vehicular driver monitoring system of claim 1, wherein the health parameter of the driver of the vehicle comprises one selected from the group consisting of (i) an emotional state of the driver of the vehicle, (ii) a drowsiness level of the driver of the vehicle, (iii) a breath analysis of the driver of the vehicle, (iv) a skin analysis of the driver of the vehicle and (v) a heart rate of the driver of the vehicle.

3. The vehicular driver monitoring system of claim 1, wherein the at least one previously determined status of the health parameter of the driver of the vehicle comprises a plurality of health parameter statuses determined by the vehicular driver monitoring system during previous trips by the driver.

4. The vehicular driver monitoring system of claim 1, wherein the vehicular driver monitoring system is operable to determine that the determined health rating of the driver of the vehicle is indicative of a threshold change in the health parameter of the driver of the vehicle.

5. The vehicular driver monitoring system of claim 4, wherein the vehicular driver monitoring system determines that the determined health rating of the driver of the vehicle is indicative of the threshold change in the health parameter of the driver of the vehicle by determining that the current status of the health parameter of the driver of the vehicle of the vehicle exceeds a threshold status of the health parameter of the driver of the vehicle.

6. The vehicular driver monitoring system of claim 4, wherein the vehicular driver monitoring system determines that the determined health rating of the driver of the vehicle is indicative of the threshold change in the health parameter of the driver of the vehicle by determining that the current status of the health parameter of the driver of the vehicle of the vehicle is less than a threshold status of the health parameter of the driver of the vehicle.

7. The vehicular driver monitoring system of claim 4, wherein the vehicular driver monitoring system determines that the determined health rating of the driver of the vehicle is indicative of the threshold change in the health parameter of the driver of the vehicle by transmitting the determined current status of the health parameter of the driver of the vehicle to a server remote from the vehicle and the vehicular driver monitoring system receiving, from the server, a response indicating that the determined health rating of the driver of the vehicle is indicative of the threshold change in the health parameter of the driver of the vehicle.

8. The vehicular driver monitoring system of claim 1, wherein the camera is disposed at an interior rearview mirror assembly of the vehicle.

9. The vehicular driver monitoring system of claim 8, wherein the camera is disposed within a mirror head of the interior rearview mirror assembly of the vehicle, and wherein the camera views through a mirror reflective element of the mirror head of the interior rearview mirror assembly of the vehicle.

10. The vehicular driver monitoring system of claim 8, wherein the ECU is disposed at the interior rearview mirror assembly of the vehicle.

11. The vehicular driver monitoring system of claim 8, wherein the ECU is disposed at the vehicle remote from the interior rearview mirror assembly.

12. The vehicular driver monitoring system of claim 11, wherein image data captured by the camera is transferred to the ECU via a coaxial cable.

13. The vehicular driver monitoring system of claim 1, wherein the ECU is operable to process data for at least one driving assist system of the vehicle.

14. The vehicular driver monitoring system of claim 1, wherein the at least one driving assist system of the vehicle comprises at least one selected from the group consisting of (i) a headlamp control system of the vehicle, (ii) a pedestrian detection system of the vehicle, (iii) a traffic sign recognition system of the vehicle, (iv) a collision avoidance system of the vehicle, (v) an emergency braking system of the vehicle, (vi) a lane departure warning system of the vehicle, (vii) a lane keep assist system of the vehicle and (viii) an adaptive cruise control system of the vehicle.

15. The vehicular driver monitoring system of claim 1, wherein the driver profile associated with the driver of the vehicle is stored in memory at a remote server that is remote from the vehicle, and wherein the vehicular driver monitoring system accesses the driver profile associated with the driver of the vehicle via wireless communication.

16. The vehicular driver monitoring system of claim 1, wherein the driver profile associated with the driver of the vehicle is stored in memory at the vehicle.

17. The vehicular driver monitoring system of claim 1, wherein the vehicular driver monitoring system generates a health notification for the driver of the vehicle responsive to the determined health rating of the driver of the vehicle being indicative of a threshold change in the health parameter of the driver of the vehicle.

18. The vehicular driver monitoring system of claim 17, wherein the health notification comprises at least one selected from the group consisting of (i) a visual alert, (ii) an audible alert and (iii) a haptic alert.

19. The vehicular driver monitoring system of claim 17, wherein the vehicular driver monitoring system provides the health notification to at least one selected from the group consisting of (i) a healthcare professional and (ii) emergency services.

20. The vehicular driver monitoring system of claim 17, wherein the threshold change in the health parameter of the driver of the vehicle comprises a threshold difference between the determined current status of the health parameter of the driver of the vehicle and the at least one previously determined status of the health parameter of the driver of the vehicle.

21. The vehicular driver monitoring system of claim 17, wherein the threshold change in the health parameter of the driver of the vehicle comprises a threshold difference between the determined current status of the health parameter of the driver of the vehicle and a predicted health parameter status, and wherein the predicted health parameter status is based at least in part on the at least one previously determined status of the health parameter of the driver of the vehicle.

22. The vehicular driver monitoring system of claim 17, wherein the vehicular driver monitoring system determines that the determined health rating of the driver of the vehicle is indicative of the threshold change in the health parameter of the driver of the vehicle by determining that the determined current status of the health parameter of the driver of the vehicle fails to satisfy at least one health parameter criteria.

23. The vehicular driver monitoring system of claim 17, wherein the vehicular driver monitoring system determines that the determined health rating of the driver of the vehicle is indicative of the threshold change in the health parameter of the driver of the vehicle by determining that the determined current status of the health parameter of the driver of the vehicle satisfies at least one health parameter criteria.

24. The vehicular driver monitoring system of claim 17, wherein the vehicular driver monitoring system determines that the determined health rating of the driver of the vehicle is indicative of the threshold change in the health parameter of the driver of the vehicle by transmitting the determined current status of the health parameter of the driver of the vehicle to a remote server and the vehicular driver monitoring system receiving, from the remote server, a response indicating that the determined current status of the health parameter of the driver of the vehicle satisfies at least one health parameter criteria.

25. The vehicular driver monitoring system of claim 17, wherein the vehicular driver monitoring system determines that the determined health rating of the driver of the vehicle is indicative of the threshold change in the health parameter of the driver of the vehicle by determining that the determined current status of the health parameter of the driver of the vehicle satisfies an unhealthy threshold status indicating that the determined current status of the health parameter of the driver of the vehicle is within an unhealthy range.

26. The vehicular driver monitoring system of claim 17, wherein the vehicular driver monitoring system determines that the determined health rating of the driver of the vehicle is indicative of the threshold change in the health parameter of the driver of the vehicle by determining that the determined current status of the health parameter of the driver of the vehicle satisfies a healthy threshold status indicating that the determined current status of the health parameter of the driver of the vehicle is within a healthy range.

27. The vehicular driver monitoring system of claim 17, wherein the vehicular driver monitoring system determines that the determined health rating of the driver of the vehicle is indicative of the threshold change in the health parameter of the driver of the vehicle by using a machine learning model.

28. The vehicular driver monitoring system of claim 1, further comprising a health sensor, the health sensor capturing health data, and wherein the vehicular driver monitoring system updates the driver profile associated with the driver of the vehicle using the captured health data and the determined current status of the health parameter of the driver of the vehicle.

29. The vehicular driver monitoring system of claim 28, wherein the health sensor comprises at least one selected from the group consisting of (i) a pollen sensor, (ii) an alcohol sensor, (iii) a heart rate sensor, (iv) a blood pressure sensor, (v) a temperature sensor, (vi) a breath rate sensor and (vii) a glucose level sensor.

30. A vehicular driver monitoring system, the vehicular driver monitoring system comprising:
a camera disposed within an interior cabin of a vehicle equipped with the vehicular driver monitoring system, the camera viewing a driver of the vehicle, wherein the camera is operable to capture image data;
wherein the camera is disposed within a mirror head of an interior rearview mirror assembly of the vehicle, and wherein the camera views through a mirror reflective element of the mirror head of the interior rearview mirror assembly of the vehicle;
wherein the camera comprises a CMOS imaging array, and wherein the CMOS imaging array comprises at least one million photosensors arranged in rows and columns;
an electronic control unit (ECU) comprising electronic circuitry and associated software, wherein the ECU is disposed at the vehicle remote from the interior rearview mirror assembly;
wherein image data captured by the camera is transferred to and processed at the ECU;
wherein the vehicular driver monitoring system accesses a driver profile associated with the driver of the vehicle, and wherein the driver profile includes status of a health parameter of the driver of the vehicle;
wherein the vehicular driver monitoring system, responsive at least in part to processing at the ECU of image data captured by the camera and transferred to the ECU, determines a current status of the health parameter of the driver of the vehicle;
wherein the driver profile associated with the driver of the vehicle comprises at least one previously determined status of the health parameter of the driver of the vehicle;
wherein the vehicular driver monitoring system updates the driver profile associated with the driver of the vehicle based on the determined current status of the health parameter of the driver of the vehicle; and
wherein the vehicular driver monitoring system determines a health rating of the driver of the vehicle based on a comparison of (i) the determined current status of the health parameter of the driver of the vehicle and (ii) the at least one previously determined status of the health parameter of the driver of the vehicle.

31. The vehicular driver monitoring system of claim 30, wherein image data captured by the camera is transferred to the ECU via a coaxial cable.

32. The vehicular driver monitoring system of claim 30, wherein the ECU is operable to process data for at least one driving assist system of the vehicle.

33. The vehicular driver monitoring system of claim 32, wherein the at least one driving assist system of the vehicle comprises at least one selected from the group consisting of (i) a headlamp control system of the vehicle, (ii) a pedestrian detection system of the vehicle, (iii) a traffic sign recognition system of the vehicle, (iv) a collision avoidance system of the vehicle, (v) an emergency braking system of the vehicle, (vi) a lane departure warning system of the vehicle, (vii) a lane keep assist system of the vehicle and (viii) an adaptive cruise control system of the vehicle.

34. A vehicular driver monitoring system, the vehicular driver monitoring system comprising:
a camera disposed within an interior cabin of a vehicle equipped with the vehicular driver monitoring system, the camera viewing a driver of the vehicle, wherein the camera is operable to capture image data;
wherein the camera comprises a CMOS imaging array, and wherein the CMOS imaging array comprises at least one million photosensors arranged in rows and columns;
an electronic control unit (ECU) comprising electronic circuitry and associated software;
wherein image data captured by the camera is processed at the ECU;
wherein the vehicular driver monitoring system accesses a driver profile associated with the driver of the vehicle, and wherein the driver profile includes status of a health parameter of the driver of the vehicle;
wherein the vehicular driver monitoring system, responsive at least in part to processing at the ECU of image data captured by the camera, determines a current status of the health parameter of the driver of the vehicle;
wherein the driver profile associated with the driver of the vehicle comprises a plurality of previously determined statuses of the health parameter of the driver of the vehicle, and wherein the plurality of previously determined statuses of the health parameter of the driver of the vehicle comprises a plurality of health parameter statuses determined by the vehicular driver monitoring system during previous trips by the driver;
wherein the vehicular driver monitoring system updates the driver profile associated with the driver of the vehicle based on the determined current status of the health parameter of the driver of the vehicle;
wherein the vehicular driver monitoring system determines a health rating of the driver of the vehicle based on a comparison of (i) the determined current status of the health parameter of the driver of the vehicle and (ii) the plurality of previously determined statuses of the health parameter of the driver of the vehicle; and
wherein the vehicular driver monitoring system is operable to determine that the determined health rating of the driver of the vehicle is indicative of a threshold change in the health parameter of the driver of the vehicle.

35. The vehicular driver monitoring system of claim 34, wherein the health parameter of the driver of the vehicle comprises one selected from the group consisting of (i) an emotional state of the driver of the vehicle, (ii) a drowsiness level of the driver of the vehicle, (iii) a breath analysis of the driver of the vehicle, (iv) a skin analysis of the driver of the vehicle and (v) a heart rate of the driver of the vehicle.

36. The vehicular driver monitoring system of claim 35, wherein the vehicular driver monitoring system determines that the determined health rating of the driver of the vehicle is indicative of the threshold change in the health parameter of the driver of the vehicle by determining that the current status of the health parameter of the driver of the vehicle of the vehicle exceeds a threshold status of the health parameter of the driver of the vehicle.

37. The vehicular driver monitoring system of claim 35, wherein the vehicular driver monitoring system determines that the determined health rating of the driver of the vehicle is indicative of the threshold change in the health parameter of the driver of the vehicle by determining that the current status of the health parameter of the driver of the vehicle of the vehicle is less than a threshold status of the health parameter of the driver of the vehicle.

38. The vehicular driver monitoring system of claim 34, wherein the camera is disposed within a mirror head of an interior rearview mirror assembly of the vehicle, and wherein the camera views through a mirror reflective element of the mirror head of the interior rearview mirror assembly of the vehicle.

39. The vehicular driver monitoring system of claim 38, wherein the ECU is disposed at the interior rearview mirror assembly of the vehicle.

40. The vehicular driver monitoring system of claim 38, wherein the ECU is disposed at the vehicle remote from the interior rearview mirror assembly, and wherein image data captured by the camera is transferred to the ECU via a coaxial cable.
